(19) United States
(12) Patent Application Publication
(10) Pub. No.: US 2024/0366190 A1
(43) Pub. Date: Nov. 7, 2024

(54) DEVICE USE-TIME ESTIMATION

(71) Applicant: Exo Imaging, Inc., Santa Clara, CA (US)

(72) Inventors: Aron COOPERMAN, Santa Clara, CA (US); Seline Andrea GEIGER, Redwood City, CA (US); Thomas TARTER, Santa Clara, CA (US)

(21) Appl. No.: 18/655,218

(22) Filed: May 3, 2024

Related U.S. Application Data
(60) Provisional application No. 63/464,438, filed on May 5, 2023.

(51) Int. Cl. A61B 8/00; G16H 40/40

(52) U.S. Cl. CPC A61B 8/58; G16H 40/40

(57) ABSTRACT

Methods and systems are provided for estimating the operational capabilities of a portable medical device, such as an ultrasound imaging device. A user may select a medical procedure, which can determine one or more operating parameters of the device. Readings from a sensor of the device can then be used to calculate a capability estimation based on the designated procedure and operating parameter(s). The capability estimation can be presented or outputted to a user, for example through a display or incorporated into a treatment program or system use parameters.

### CROSS-REFERENCE TO RELATED APPLICATIONS

This application claims the benefit of U.S. Provisional Application No. 63/464,438, filed May 5, 2023, the entirety of which is incorporated herein by reference.

### FIELD OF THE INVENTIONS

The present disclosure generally relates to systems, methods, and devices for estimating the remaining amount of time that an electronic medical device may be used before a shut-off parameter is reached.

### BACKGROUND

Ultrasound imaging is an imaging method that uses sound waves to produce images of structures within a patient's body. Because ultrasound scans are captured in real-time, they can also show movement of the body's internal organs as well as blood flowing through the blood vessels. The images can provide valuable information for diagnosing and directing treatment for a variety of diseases and conditions.

Electronic devices have several known limitations, including limited battery power and a maximum heat at which the device can successfully operate. Battery-powered electronic devices can only operate for a limited about of time before running out of battery. If a procedure is known to take a set amount of time, and an electronic device is known to have remaining battery power such that the device will be able to operate for less than that set amount of time, that electronic device cannot be used to complete that procedure. Similarly, if an electronic device heats up while it is being used, it can only be used for so long before it may reach a maximum temperature, past which the device cannot continue to operate without problem.

### SUMMARY

The systems, methods and devices of this disclosure each have several innovative aspects, no single one of which is solely responsible for the desirable attributes disclosed herein.

Portable (e.g., handheld, and/or battery-operated) ultrasound devices can provide greater case of use and flexibility than traditional cart-based or wired ultrasound systems. However, in accordance with at least some embodiments disclosed herein is the realization that improvements in portability and case-of-use do not contemplate the problems addressed by the present disclosure or otherwise enable the unique and vital improvements presented herein.

For example, in accordance with at least some embodiments disclosed herein is the realization that for certain procedures or in certain clinical settings, the use of portable ultrasound devices may complicate patient care due to battery life and overheating issues. However, the innovative principles disclosed herein, in accordance with some embodiments, can enable the user of a portable ultrasound device or system of devices to minimize occurrences of overheating or running out of battery power while performing a medical procedure. In some embodiments, the device or system can comprise a predictive modeling component, an operational capability estimator, and/or unique feature monitoring equipment that can assist the user in planning and performing one or more procedures for one or more patients.

The present disclosure provides a system and related methods whereby the remaining amount of time that an ultrasonic imaging probe may be used before running out of battery, overheating, or reaching other critical levels of performance can be calculated and communicated to the user of the imaging probe.

For example, an algorithm may take in readings from various sensors in, on, and around the imaging probe. The algorithm may also take in information or operational settings input or set by the user of the probe. The algorithm may use prior stored data, current readings or signals from the probe, and/or other information accumulated regarding the imaging probe.

The algorithm may predict a length of time that the probe can used before the probe needs to cool down, recharge, receive maintenance, or otherwise need to pause its operation. This prediction may be updated in real time due to changing operational parameters. This prediction may be presented to the user of the probe or other relevant person through a communication interface or through other auditory, visual, haptic, or other feedback. The algorithm may also consider other similar imaging probes within a system of imaging probes and recommend the use of one or several probes within the system.

As disclosed herein, in some embodiments, a method for estimating the operational capabilities of an ultrasound imaging device can comprise receiving a selection of a medical procedure and determining an operating parameter of the imaging device based on the selected medical procedure. Operational data from a sensor of the imaging device can then be used to calculate a capability estimation. The capability estimation can be presented or outputted to a user, for example through a display or incorporated into a treatment program or system use parameters (such as a preplanned arrangement, order, or prescribed use of one or more ultrasound devices of a system).

Some embodiments of the methods disclosed herein may be performed at a computing device that includes one or more processors and memory associated with at least one ultrasound imaging device.

Further, in accordance with some embodiments, the capability estimation may be related to the selected medical procedure(s) to be performed using the imaging device(s), and it may be based on at least one operating parameter and at least one operational data set.

In some embodiments, a system comprising at least one ultrasonic imaging probe and an electronic device can be used by a clinician to complete medical procedures on a patient. These procedures may include, for example, ultrasonic imaging scans. The clinician may be able to select, via the device or via the probe itself, which procedure they wish to complete and a variety of settings related to the procedure or the operation of the device.

Optionally, one or more components of the system, such as the ultrasound imaging probe(s) and/or the electronic device(s) (which may be collectively or individually referred to herein as the “system,” along with the software and/or hardware associated therewith), can operate or serve up a program that can be utilized to facilitate management of the device or system. The program can include an algorithm that can receive data from one or more sensors within the probe(s) and/or electronic device(s), such as temperature, time, battery level, battery power level, and/or other operational parameters. The algorithm may then be able to use this data to predict whether the probe(s) (and/or device(s)) should be able to complete the selected procedure(s).

In accordance with some embodiments is the realization that a probe(s) may not be able to complete a selected procedure(s) for a variety of different reasons, some of which may not be easily ascertainable or discernible by the user prior to initiating the procedure(s). For example, before beginning a selected procedure(s), the probe(s) may be at an elevated temperature. This may be due to a variety of reasons, including due to, for example, having recently been used for another, prior procedure, or due to an elevated ambient temperature of the probe's environment that may cause the probe(s) to overheat or become uncomfortable for the patient before the selected procedure(s) can be completed. Additionally, for example, in some embodiments, before beginning a selected procedure, the probe(s) may have a certain battery charge or level that may be insufficient to perform the selected procedure, and thus the probe(s), device(s) or system may predict that the probe(s) or device(s) may run out of battery power before the procedure can be completed.

Optionally, in accordance with some embodiments, devices and systems disclosed herein can also provide a real-time monitoring that reflects data from one or more sensors of the probe(s) and/or device(s) as the selected procedure(s) is being performed and which might affect the performance of the selected procedure(s). As discussed above with regard to the example parameters or data, the real-time monitoring can provide ongoing feedback and if necessary, prompt a change from one probe or device to another, based on the real-time monitoring and implementation of an algorithm based on parameters and/or data from a probe, device, and/or system.

For example, in some embodiments, a maximum or shut-off temperature exists, past which a probe cannot or should not operate for safety or device longevity reasons. When the probe reaches this maximum temperature, it may automatically shut-off. This may stop the probe from reaching a temperature that would be too high and potentially cause damage to the internal electrical components of the probe and/or cause discomfort to a patient.

With the system being able to predict when the probe(s) and/or device(s) will and won't be able to complete a selected procedure, the user can initiate a procedure with confidence that their probe will be able to complete the procedure. Further, the user can have confidence that a single probe will be appropriate to use and/or will be able to be exchanged with another probe to permit the user to complete a procedure without having overheating or battery life issues in the middle of the procedure. Accordingly, the present disclosure provides advantageous peace of mind and improves patient care by minimizing discomfort to the patient and minimizing delays due to restarting the procedure or interchanging hardware during the procedure.

In some embodiments, the algorithm calculates a heating curve for a probe. The heating curve can be calculated using real-time data. Further, some embodiments can also use historical, recorded temperatures from prior and/or other probes and/or other scans, including prior scans of the same type of procedure as the current procedure(s), as well as scans that are different from the selected procedure(s). The algorithm can calculate or use a time constant, t, which can be used along with real-time temperature data collected from sensors within the probe, to estimate how long the probe can be used before it reaches its maximum/shut-off temperature. The time constant may be calculated using a lumped capacitance model.

In some embodiments, the algorithm can optionally calculate a cooling curve for a probe by using real-time and/or historical, recorded temperatures from other probes and/or other scans, including prior scans of the same type of procedure as the current procedure(s), as well as scans that are different from the selected procedure(s). The algorithm may use this recorded data, along with real-time temperature data collected from sensors within the probe, to estimate how long the probe needs to cool before it can be used to complete a selected procedure.

In some embodiments, the algorithm may be able to predict when a probe will be able to complete a selected procedure. If the algorithm determines that the probe will not presently be able to complete the procedure as selected by the user, the algorithm may be able to calculate and/or predict the time until or at which the probe will be able to complete the procedure. This may include calculating how long the probe needs to charge before having enough battery power to complete the procedure, or how long the probe needs to rest or cool down before the probe will be able to generate heat during the course of the procedure while still remaining below the maximum/shut-off temperature.

Further, in some embodiments, this prediction may include a recommended maintenance or repair procedure based on data associated with detected operational data regarding scan quality or hardware issues. The algorithm may be able to compare the collected, real-time data with a calculated expected curve or model. If the real-time data deviates past a determined amount from the predicted data, it may be inferred that the probe requires maintenance, repair, or other consideration. Further, the algorithm may be able to output this maintenance requirement such that the user of the probe is alerted, including, for example, communicating an alert display to the electronic device or communicating a form of visual, auditory, or haptic feedback to be sent by the probe itself.

In some embodiments where the clinician has access to a system of probes, the system can calculate and then display to the user which of the probes could complete the procedure. For example, the system could indicate to the user when each of the probes will be able to complete the procedure and/or how much they each need to charge or cool before use.

In accordance with at least some embodiments disclosed herein is the realization that a correctly functioning medical device is critical to correctly completing medical procedures and providing correct information to patients. As disclosed herein, some embodiments can provide a method for estimating replacement time or providing diagnostic capability estimation for a component of an ultrasound imaging device. For example, the method can comprise determining an operating parameter of the component, receiving operational data from a sensor of the imaging device, the operational data being representative of the operating parameter of the component, calculating a service estimation based on the operating parameter and the operational data, and outputting the service estimation to a user for assisting in replacement or repair of the component of the imaging device. This method may be accomplished at a computing device that includes one or more processors and memory associated with the ultrasound imaging device.

Accordingly, some embodiments can provide a method that enables a diagnostic capability. Therefore, in some embodiments, this diagnostic capability may allow a clinician to easily know when to replace various components of the probe. For example, ultrasonic imaging devices may use transducer arrays, batteries, and other electronic components that can wear-out over time. Some embodiments disclosed herein can enable a clinician to be assured that their probe is operating within set parameters when the algorithm returns favorable service estimations. The clinician may be notified as soon as a component no longer functions as it should, and therefore the clinician may operate confident in the knowledge that their probe is functioning correctly.

In some embodiments, an ultrasound imaging system may comprise one or more medical imaging devices each having a processor and a sensor, and a controller device having a program stored in memory. The program may comprise instructions for receiving a selection of one or more medical procedures, determining an operating parameter of each of the one or more medical devices based on the selection of one or more medical devices, receiving operational data from the sensor of each of the one or more medical devices, calculating a capability estimation related to the selection of one or more medical procedures to be performed using the one or more medical devices and based on the operating parameter and the operational data of each of the one or more medical devices, and outputting the capability estimation to a user.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 shows a schematic diagram of a clinician using an ultrasonic imaging probe to complete a procedure on a patient, while viewing information on an electronic device, according to some embodiments of the present disclosure.

FIG. 2 shows a schematic diagram of an ultrasonic imager, according to some embodiments.

FIG. 3 shows a top view of a transceiver tile, according to some embodiments.

FIG. 4 shows an example user interface of a single-probe system, according to some embodiments.

FIG. 5 shows an example user interface of a multi-probe system, according to some embodiments.

FIG. 6 shows a schematic diagram of a user interface interacting with a multi-probe system, according to some embodiments.

FIGS. 7A-7F show example user interface displays, according to some embodiments.

FIG. 8 shows a graph displaying temperature over time for a probe, according to some embodiments.

FIG. 9A shows a graph displaying temperature and power of a probe over time for a probe, according to some embodiments.

FIGS. 9B-9D show example user interface displays, according to some embodiments.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for case of understanding.

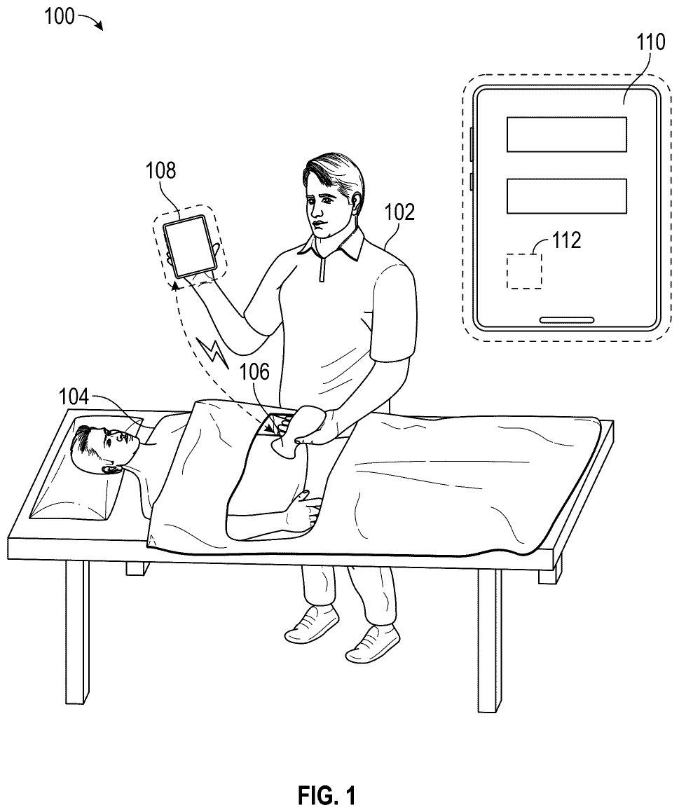

FIG. 1 shows a system 100 that a clinician 102 can use to perform a medical procedure on a patient 104. The clinician 102 may use a medical device, such as a probe 106, to complete the medical procedure on the patient 104. The clinician 102 may further use an electronic device 108 to assist in completing the medical procedure. The device 108 can interact with the probe 106, such as by controlling an aspect of the probe 106, displaying information from the probe 106, or otherwise communicating with the probe 106 to send or receive information or commands that can be displayed and/or executed during the course of the procedure.

In some embodiments, the device 108 may comprise a user interface 110. The user interface 110 may include various graphics and displayed information that may help the clinician 102 in their use of the probe 106. The device 108 may further be used to change or control various aspects of the probe 106. The device 108 may further include memory 112. The memory 112 within the device 108 may be used to store and run an application or program that controls the user interface 110.

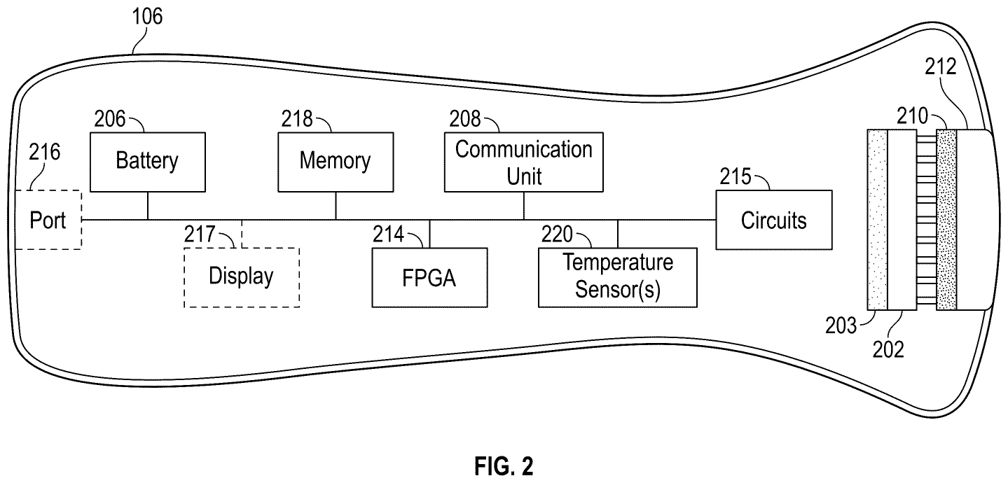

FIG. 2 shows a schematic diagram of the probe 106, according to some embodiments. In some embodiments, the probe 120 may be an ultrasonic imaging device.

As depicted in FIG. 2, the probe 106 may include transceiver tile(s) 210 for transmitting and receiving pressure waves. A coating layer 212 may operate as a lens for steering the propagation direction of and/or focusing the pressure waves and may also function as an impedance interface between the transceiver tile 210 and the skin of the patient 104.

The probe 106 may further include a control unit 202, such as an application specific integrated circuit chip (hereinafter “ASIC”), for controlling the transceiver tile(s) 210 and coupled to the transceiver tile 210 by bumps or other suitable connections. Field Programmable Gate Arrays (FPGAs) 214 may control the components of the probe 120. Circuit(s) 215, such as an Analogue Front End (AFE), may exist for processing/conditioning signals.

An acoustic absorber layer 203 may absorb waves that are generated by the transceiver tiles 210 and propagate toward the circuit 215.

The probe 106 may further include a communication unit 208 for communicating data with an external device, such as the electronic device 108 and/or other devices. Communication between the probe 106 and other devices may be possible through one or more ports 216. The probe 106 may also comprise a speaker, microphone, and/or other equipment for permitting communication and prompts to the clinician 102.

The probe 106 may further include a memory 218 that can store data. Further, the probe 106 may further include a battery 206 to provide electrical power to the components of the imager.

Optionally, the probe 106 may include a display 217 for displaying images of, for example, ultrasonically scanned target organs.

In some embodiments, the electronic device 108 may have a display/screen. In some embodiments, the display can advantageously be incorporated into the electronic device 108 instead of or in addition to some portion of the probe 106. However, the system can be implemented to permit modular operation, viewing, and/or control of the probe and its operation. For example, the display generated from the probe 106 can be separate from the probe 106 itself and optionally, even separate from a device or controller that the clinician 102 can use to control one or more functions or features of the probe 106.

In some embodiments, the probe 106 may receive electrical power from the electronic device 108 through one of the ports 216. In such a case, the probe 106 may not include the battery 206. It is noted that one or more of the components of the probe 106 may be combined into one integral electrical element. Likewise, each component of the probe 106 may be implemented in one or more electrical elements.

In some embodiments, the clinician or other personnel may apply gel on the skin of the patient 104 before the skin makes a direct contact with the coating layer 212 so that the impedance matching at the interface between the coating layer 212 and the skin of the patient 104 may be improved. The transceiver tiles 210 may be mounted on a substrate and may be attached to an acoustic absorber layer. This layer can absorb any ultrasonic signals that are emitted in the reverse direction, which may otherwise be reflected and interfere with the quality of the image.

As discussed below, the coating layer 212 may comprise a flat matching layer to facilitate the maximization of the transmission of acoustic signals from the transducer to the body and vice versa. In some embodiments, the thickness of the coating layer 212 may be a quarter wavelength of the pressure wave generated by the transceiver tile(s) 210. Beam focus in the elevation direction, which can be along the direction of the length of the column, can be electronically implemented in control unit 202. Even then, the lens may be designed with a focus in some cases. The probe 106 may use the reflected signal to create an image of an organ of the patient 104 and results may be displayed on a screen in a variety of format, such as graphs, plots, and statistics shown with or without the images of the organ.

In some embodiments, the control unit 202, such as an ASIC, may be assembled as one unit together with the transceiver tiles. In other embodiments, the control unit 202 may be located outside the probe 106 and electrically coupled to the transceiver tile 210 via a cable. In some embodiments, the probe 106 may include a housing or enclosure that encloses the components of the probe 106 and a heat dissipation mechanism for dissipating heat energy generated by the components.

The probe 106 may also include one or more temperature sensors 220. For example, one or more temperature sensors 220 may be located at or near the battery 206, in order to monitor the battery temperature. Further, one or more temperature sensors 220 may be located near the circuits 215 to monitor the temperature of the circuits. Furthermore, one or more temperature sensors 220 may be located elsewhere within the probe or located on the exterior of the probe 106. Exterior temperature sensors may monitor ambient temperature, temperature of the hand grip of the clinician 102, or other possible temperatures that may affect the overall operation of the probe 106.

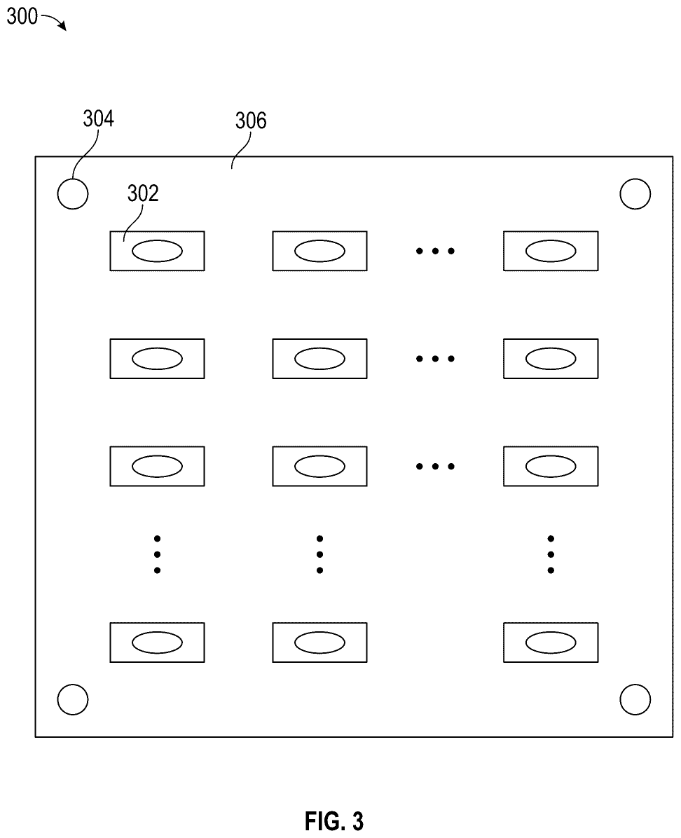

FIG. 3 shows a top view of a transceiver tile 300 according to embodiments of the present disclosure. It should be apparent to those of ordinary skill in the art that the probe 106 may include any suitable number of tiles and the tiles may be arranged in any suitable manner, and each tile 300 may include any suitable number of piezoelectric elements 302 that are disposed on a transceiver substrate 306. On the substrate 306, one or more temperature sensors 304 may be placed in order to monitor the temperature of the transceiver tile 300 during operations. In some embodiments, the transceiver tiles 300 may be micromachined elements fabricated from a substrate.

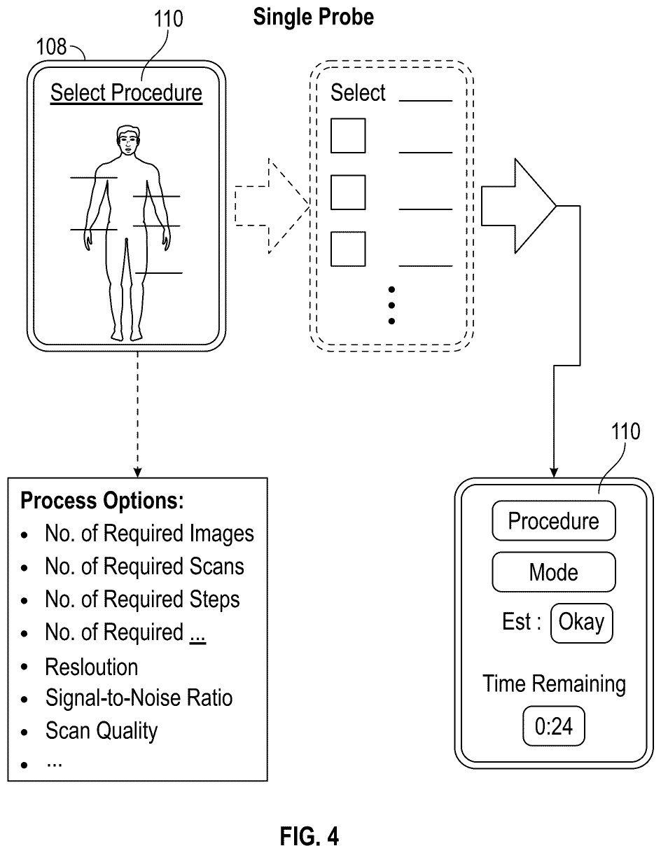

FIG. 4 shows an example user interface, or UI, according to some embodiments, in which a single probe is available to be used. The probe 106 may be used by the clinician 102 to perform a medical procedure on a patient 104. The probe 106 may have a connection to an electronic device 108. This connection may allow the probe 106 and the device 108, along with any programs or algorithms stored on the probe 106 or the device 108, to continuously share data and other information between the probe 106 and the device 108. Via the UI, the device 108 may be used to control one or more settings, functions, actions, or other aspects of the probe 106. These settings, other aspects, and controls may also be able to be controlled from the probe 106 itself, for example through buttons, dials, switches, or other indicators on the probe 106.

The device 108 may have a display screen that shows a user interface 110 that can be viewed by, for example, the clinician 102. The user interface 110 may have a screen that allows the user of the probe 106, such as the clinician 102, to select a procedure to perform on the patient 104. The user interface 110 may include various screens, including, for example, a screen showing an example human patient displaying the various locations a medical procedure may be performed using the probe 106.

In some embodiments where the probe 106 is an ultrasonic imaging device, available procedures may include, for example, cardiac scans of the patient's heart, scans of the patient's lungs, kidneys, or other internal organs, vascular scans, OB/GYN-related scans, or other possible procedures that may be performed using ultrasound imaging.

After selecting a procedure, the user interface 110 may display information via a screen layout that displays/confirms the selected procedure and/or allows selection of other device settings or criteria available to the clinician 102 for controlling one or more functions of the probe and/or aspects of the selected procedure.

The user interface 110 may then display, to the clinician 102 or any other person viewing the device 108, a screen confirming the procedure or operation selected as well as any modes or other settings that have been selected.

As discussed herein, in some embodiments, the user interface can display an indication of whether the probe is capable of performing the selected procedure, with the selected criteria, at the present moment. For example, one or more components of the system (e.g., the probe and/or the device) can comprise an algorithm that can consider one or more factors and provide a recommendation or determination regarding a capability of the system, such as a capability of the probe or device.

For example, the algorithm may also be able to calculate whether the probe 106 will be able to perform the selected procedure, with the selected criteria, at a later point in time. Such a later point in time may be after the probe 106 has charged, increasing its available battery power, or after the probe has been at rest, allowing the probe 106 to cool down in temperature.

Optionally, after the clinician 102 or any other person has selected the procedure to be performed, the algorithm may further calculate the readiness of the probe as according to various process options that are related to the selected procedure. These process options that may be considered by the algorithm may include, for example, the number of required images that must be successfully completed in order to complete the selected procedure, the number of required scans that must be completed to successfully complete the selected procedure, the number of required steps from beginning to end that are necessary to successfully complete the selected procedure, or other requirements, such as what settings have been selected, including quality settings, resolution settings, noise settings, or other settings related to the selected procedure or the operation of the probe.

Further optionally, after the algorithm has calculated the readiness of the probe with respect to the selected procedure and various other procedure or probe related criteria, the user interface 110 may indicate to the device user whether the algorithm has determined that the probe should be able to complete the procedure, or is “okay” to use, or whether the probe has been calculated to not be able to complete the selected procedure, or is “not okay.” The display examples shown in FIG. 4 are illustrative of one possible display, and it should be recognized that other possible display formats exist. In some embodiments, for example, the UI may show “okay” and “not okay” to indicate whether the probe should be able to complete the inputted/selected procedure. In some embodiments, the display may show “ready” or “needs to charge” or “needs to cool down” or other similar words or phrases indicating the status of the probe. In some embodiments, the display may show a check mark or an “X” or other similar graphics or words to indicate whether the probe should or should not be able to complete the procedure. In some embodiments, the display may utilize colors, such as green to indicate readiness and red to indicate an issue.

The user interface 110 may also display information to the user of the device 108 and/or the user of the probe 106, such as the calculated time remaining that the probe 106 should be able to operate at its current settings. This “estimated use-time” may be displayed in a count-down timer, a clock, graphics showing changing remaining levels, or other visual or auditory cues. Optionally, the user interface 110 can also display information such as the calculated time remaining that the probe 106 could function if certain settings are changed, as well as display which and how certain settings should be changed.

In some embodiments, the user interface 110, in displaying information to the clinician 102 or other user of the device 108, may allow this user to select a procedure to complete by showing the user a list of all available procedures, different icons displaying different procedures, or other visual ways of describing every available procedure.

The user interface 110 may include various guidance screens to direct the user of device 108 to input other required information. In some embodiments where the probe 106 is an ultrasonic imaging device, this information may include selecting various settings of the probe 106, such as different available excitation modes including low, medium, and high wattage settings, scan depth, gain values, and contrast levels.

The user interface 110 may visually direct the user of device 108 to particularly important information or cue the user to whether a result is desirable or not. This may include, for example, displaying “okay” or similar wording in green but displaying “not okay” or similar wording in red. The user interface 110 may also use various common indicator colors in the display of the calculated information, such as green for desirable results, orange or yellow for warnings, and red for errors or undesirable results.

In some embodiments, the algorithm may run continuously any time the probe 106 and/or the device 108 is in use. However, the algorithm can also automatically run only at start up, shut down, and/or when initiated or prompted by the user.

If running continuously, the algorithm may be running in the background of the user interface 110 such that the viewer of the interface is unaware that the algorithm is currently running. Alternatively, the user interface 110 may include some indication that the algorithm is currently running, such as showing updating progress bars, continuously running arrows, or other visual indications that the algorithm is currently processing information. In these embodiments, the algorithm may continuously monitor data relating to the probe 106 and may continuously update the prediction results.

If running at discrete or selected times during use, the algorithm may run and complete its initial calculations and/or predictions before the clinician or other user of the probe begins using the probe. In some embodiments, the algorithm may be run at a later time before, at the end of, or during the use of the probe, and the results may be updated. Further, the algorithm can optionally be triggered or initiated by the user at any time during use or it can otherwise be programmed to automatically run at selected times, such as at the beginning and/or end of a procedure or at discrete intervals during a procedure.

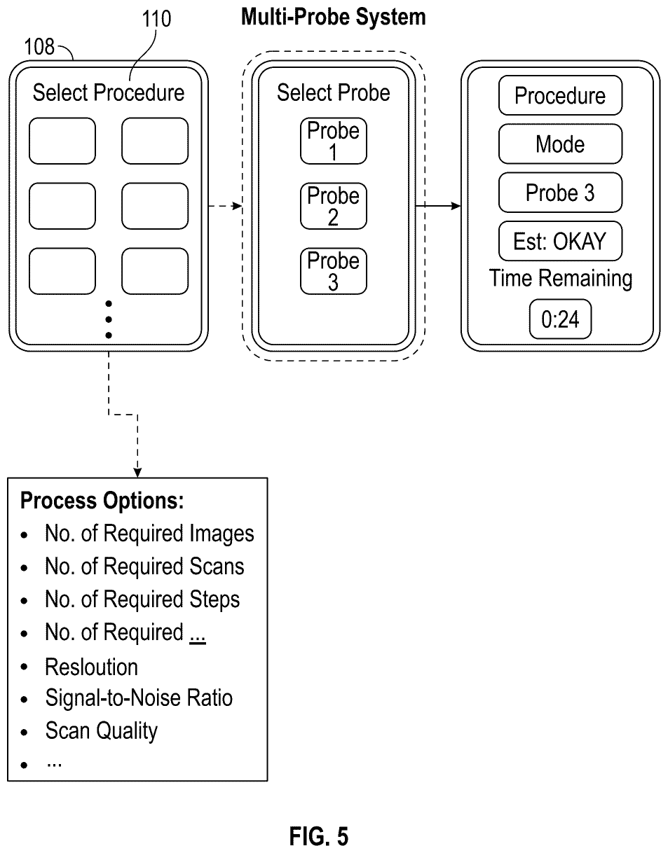

FIG. 5 shows an example user interface, or UI, according to some embodiments in which a system of probes is available to be used. The system of probes includes two or more probes that are each able to be monitored. This monitoring may include monitoring battery level and monitoring internal and ambient temperatures. Similar to FIG. 4, discussed above, the UI shown in FIG. 5 may display a series of screens to the clinician 102 or any other person using the probe 106 or any other probes in the same system of probes as the probe 106. The UI may guide the probe user to select a series of options, including what procedure the clinician or user wants to perform and what mode they want to operate the probe in. Mode options may include, for example, low, medium, or high wattage settings. The user may also be able to select which probe in the system of probes that they want to use.

In some embodiments, an algorithm may calculate/predict whether the selected probe will be able to complete the selected procedure according to the selected parameters and other factors. Other factors the algorithm may consider include the temperature of the probe, the ambient temperature, the remaining battery life of the probe and the remaining battery life of the electronic device or other system components, and temperature, battery, or other sensor data acquired from prior procedures (whether the same procedure or a different procedure). This data may be factored into the algorithm using exact values or using averages, projections, or estimates based on the data.

In the algorithm's calculations, a variety of process options may be considered. In some embodiments where the probe 106 is an ultrasonic imaging device, these process options may include factors such as the number of images required to complete the selected procedure, the number of required scans necessary to complete the selected procedure, the number of required steps to successfully complete the selected procedure, or any other quantitative values to consider. The process options may also include resolution settings, minimum signal-to-noise requirements, scan quality requirements, or any other settings related to the output of the probe.

The UI may then display the calculations and/or predictions to the user of the device 108, on which the UI is shown. The results may include a screen detailing to the user what procedure has been selected, what mode or other settings have been selected, which probe the user has indicated they wish to use, and whether the algorithm has determined this probe should be able to complete the procedure as detailed. The UI may also display to the user a timer showing the remaining amount of time that the selected probe should be able to operate at its current settings.

In some embodiments where the algorithm runs continuously and updates the predictions routinely, a countdown timer may update to reflect changes in the monitored systems that affect the predicted amount of time the probe will be able to function before running out of power or overheating.

For example, if the ambient temperature of the room increases during a procedure, the countdown timer may drop more than a minute during a time frame of sixty seconds to reflect the effect of the increased ambient temperature. In another example, if the clinician begins a procedure but then takes a break in the middle of the procedure, the algorithm will be able to update its countdown timer with an increased time-remaining prediction, as the probe at idle will use less battery and generate less heat than the probe in use.

In some embodiments, the UI may guide the user to first select which procedure they wish to complete and any mode settings relevant to this procedure. Thereafter, the UI may display to the user which of the probes in their available system of probes should be able to complete the procedure as inputted by the user. The UI may indicate to the user which of the probes in the system of probes are not currently able to complete the procedure. Optionally, the UI may indicate when those probes may be able to complete the procedure at a later time. For example, the UI may indicate whether a probe needs to cool down and how long that is estimated to take, or, the UI may indicate if a probe needs to charge and/or how long of a charge time is calculated to be necessary before the probe is ready to complete the procedure as inputted.

In some embodiments, the user may be able to first input what procedure they wish to complete and any other parameters. Thereafter, the algorithm may connect to at least one or all of the available probes, consider each probe's monitored temperature, battery life, and/or other factors, and run the algorithm to predict whether each probe will be able to complete the procedure. Those results may then be displayed to the viewer of the UI.

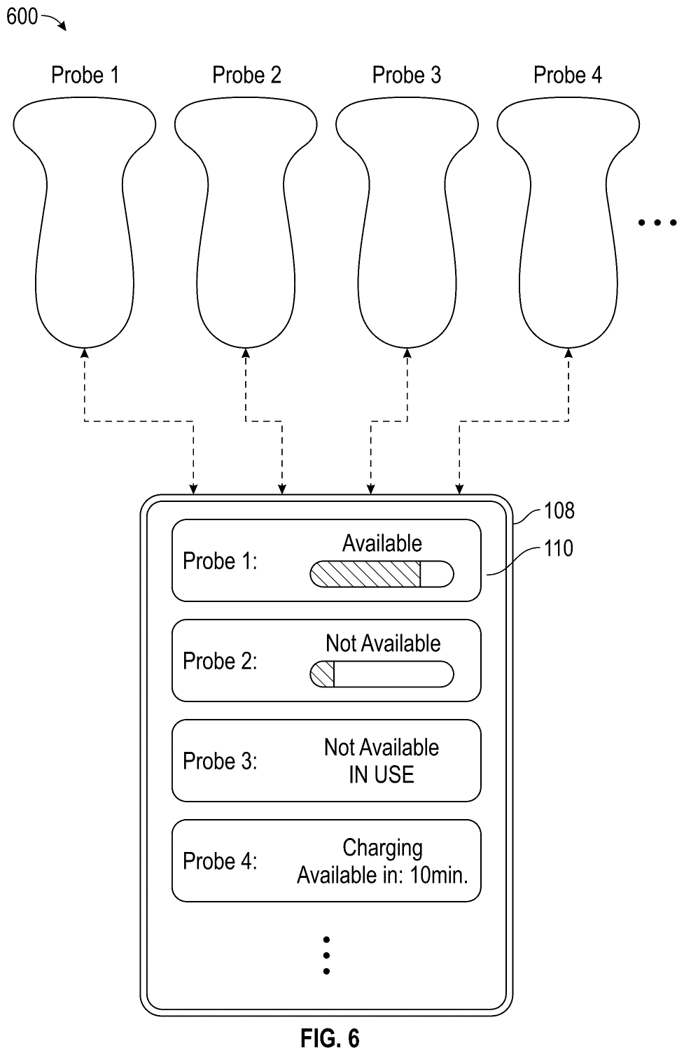

FIG. 6 shows an example UI 110 and electronic device 108 according to some embodiments. The electronic device 108 may be able to remotely connect to a system of probes 600. Through this remote connection, the electronic device may be able to access information relating to at least one or all of the probes within the system of probes 600. This information may include, for example, the internal temperature of each device, including the temperature at or near heat-generating electrical components such as transducers and/or a battery. This information may additionally include the ambient temperature around each probe and the battery level of each probe. This information relating to each probe may be used by an algorithm to predict whether each probe will be able to complete a specified medical procedure.

The UI 110, in some embodiments, displays to a viewer of the interface whether or not each probe in the system of probes 600 is available for use in completing the selected procedure. The UI may indicate whether is each probe is available or not available. This display may include words, graphics or other symbols, or a combination of display types to indicate to the user whether each probe is available for their needs or not. This display, in some embodiments, may also include graphics such as fully filled, partially filled, or empty progress bars to indicate what percentage of “life” each probe has been calculated to have left.

In some embodiments, this display within the UI 110 may also explain why a certain probe is not available, such as showing whether a probe is already in use by another person with access to the same system of probes 600, or whether a probe's current temperature is too high already or its battery level is too low to complete the procedure before running out of power or generating too much heat. In instances such as these, the algorithm may be able to calculate and display to the user when the probes that are not currently available will be available. This may include, for example, calculating and displaying how long a probe needs to charge or how long a probe needs to cool down before it could be used to complete the specified procedure.

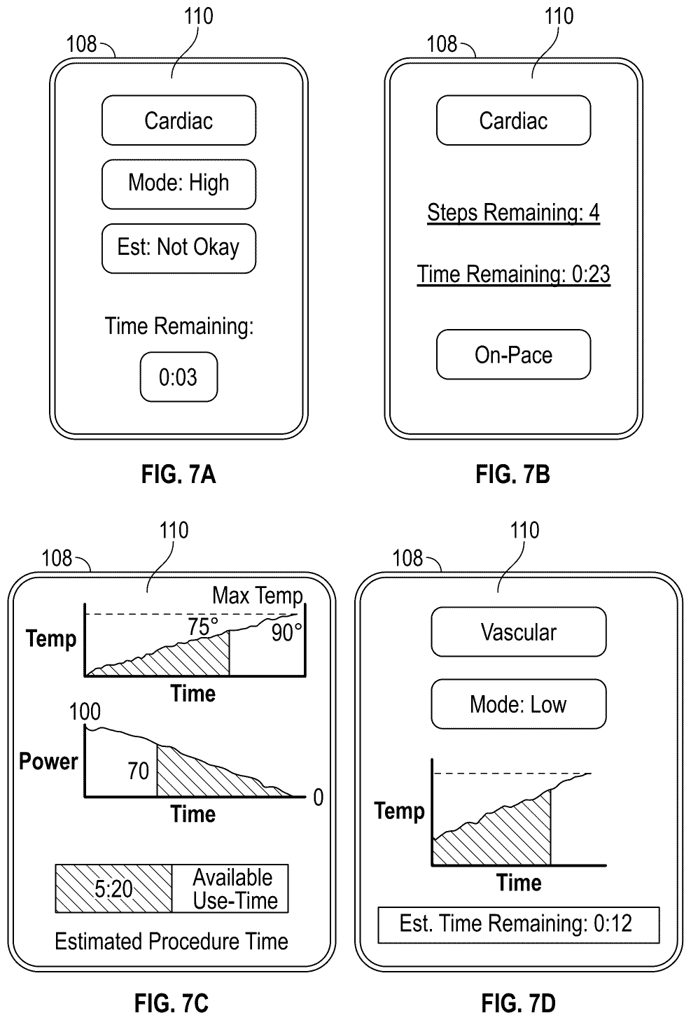

FIGS. 7A-7F illustrate example user interfaces 110 that may be displayed on the electronic device 108. These UIs may display various sets of information to the clinician using the probe 106 or any other person viewing the user interface.

As shown in FIG. 7A, the UI 110 may display, for example, which medical procedure type the clinician has selected, which operational mode type has been selected, and whether the selected probe has been predicted to be able to complete the procedure as selected. The UI 110 may also include graphics, such as a countdown timer that displays the remaining amount of time that the probe being used should be able to be operated at its current levels before running out of battery or overheating. This UI 110 may further include other graphics, colors, sounds, haptic feedback felt within the device 108, or other indications to the clinician or other viewer or user of the device 108 as to the current status of the probe being used.

In some embodiments, the estimations/predictions, the time remaining countdown, or other displayed information that is a calculated result of the algorithm may update as needed during the course of the procedure. The algorithm may routinely or constantly be given data regarding the probe being used, such as battery and temperature levels. If, for example, one of these monitored factors changes at a rate that is different than a predicted value, the algorithm may update the displayed results accordingly. For example, a probe may indicate a current ambient temperature before the procedure is begun. During the course of the medical procedure, however, the ambient temperature may increase or decrease. An increase in ambient temperature may decrease the amount of time it will take the probe to reach its maximum/shut off temperature. A decrease in ambient temperature may extend the amount of time the probe will be able to be used before it reaches its maximum/shut off temperature. Therefore, the algorithm may note this change in ambient temperature, update the predicted amount of time the probe will be able to be used before overheating, and update the displayed results.

FIG. 7B displays an example user interface 110, according to some embodiments, including various potential pieces of information that may be displayed on the UI. If the program controlling the UI has been programmed to track certain aspects of a medical procedure, the UI 110 may display information regarding these aspects to the user.

For example, if a certain procedure has a known number of steps, the program may track how many of these steps have been completed and display how many steps remain to be completed. If the program includes data regarding how long each of these steps tend to take to complete, on average, then the program may be able to track whether the clinician or user of the probe 106 is “on pace” to complete the selected procedure in a standard amount of time. A “pace tracking” function may be useful for internal metrics, such as letting a clinician know whether they are completing certain procedures in a standard amount of time. This pace tracking may also be useful for insurance purposes, such as tracking whether procedures are completed in correct amounts of time for the procedures to be completed correctly or to match clinician billing times to submitted completed procedures.

FIG. 7C displays an example UI 110 according to some embodiments. The UI 110 may be able to display information about the probe being used through various graphical formats. This may include showing the current temperature of the probe in relation to how close the probe is to its maximum/shut off temperature or displaying how much battery power the probe has left relative to a full charge. This may also include displaying what the algorithm has calculated the estimated time it will take the clinician to complete the selected medical procedure to be, and this may also include displaying how much of that time has already passed. These various example graphical displays may update in real time as the medical procedure is being completed.

FIGS. 7D and 7E show example user interfaces 110 according to some embodiments. The user interfaces 110 may include various combinations of the previously described possible displays.

In some embodiments, certain metrics may be used to discover possible issues with the functioning of the probe 106. For example, the program running the user interface 110 may include in its program memory programmed or calculated information regarding ideal probe function. This information may be relevant to, for example, how long it should take the probe, operating at a set mode and in a known ambient temperature, to reach the probe's maximum/shut-off temperature. If the true operation of the probe, as tracked by the program and/or algorithm during operation of the probe, differs outside of a set tolerance from this idealized time frame, then it could be inferred that the probe is not operating at peak performance. This may include, potentially, that some component of the probe should be replaced. Accordingly, in some embodiments, the system can provide a “diagnostic” feature that can give insight into the performance status of the probe. This feature could allow the probe to be replaced or upgraded as necessary. This diagnostic feature could easily allow clinicians to be assured that their probe is operating correctly, and it could easily allow overseers of the probe or system or probe to know when the probe needs to be upgraded or replaced.

In some embodiments, the UI may show the selected settings and the predicted results. In some embodiments, the UI may show the selected settings and the predicted results and may also allow the user of the device 108 to change the selected settings from this display screen. For example, the UI may show that the user has selected an OB/GYN procedure and selected to operate the probe in a “high” power setting. From this same display screen, the user may be able to then click the “high” power selection and change it to “low” power. The UI may then be able to update the display with the updated results of the prediction, as calculated by the algorithm.

FIG. 7F shows an example UI 110, according to some embodiments. As discussed, the UI may be able to display calculated information to the user via a combination of words and graphical displays that update in real-time. These graphical displays may include progress bars that empty or fill based on the criteria being displayed and the progress of the procedures. For example, a bar may fill as the probe heats up during a procedure to show how close the probe is to reaching maximum/shut-off temperature. A bar may empty as the battery of the probe is used during the procedure to show how close the probe is to running out of battery power. These calculated values may then be used by the algorithm to display a predicted overall use-time that is available for the probe. This too may be displayed, for example, in a progress bar that empties as the probe is used.

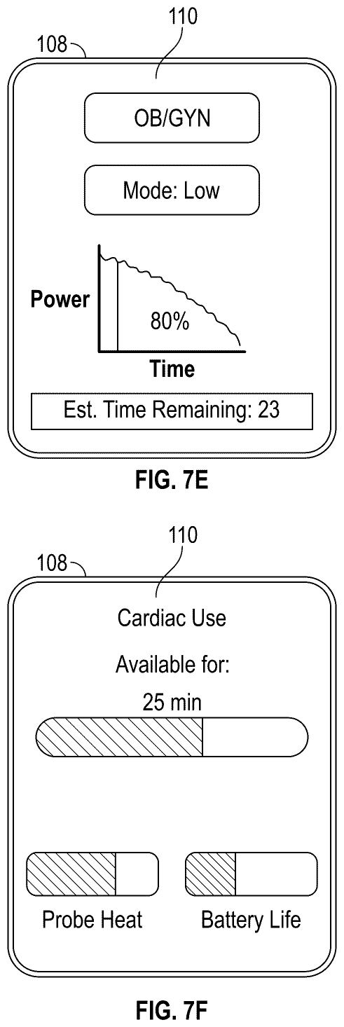

FIG. 8 shows an example plot 800 that illustrates how the temperature of a probe may change over time during a variety of actions. These actions may include different procedures, charging the probe, and the probe remaining at rest.

In some embodiments, the probe is an ultrasonic imaging device and includes transducers. These transducers may be piezoelectric micro-machined ultrasonic transducers (PMUTs). These PMUTs may generate heat while in use, causing the temperature of the probe to increase.

While the probe is being used, the internal temperature of the device will increase. The probe may have a pre-programmed maximum or shut-off temperature. When the probe reaches this shut-off temperature, the probe may automatically turn off. This automatic turnoff feature may help prolong the life of the probe, as allowing the probe to overheat past a certain temperature could cause damage to components of the probe.

In some embodiments, this shut off temperature may consider the temperature at which continued use of the probe becomes uncomfortable to the patient. As ultrasonic imaging devices are typically applied to the skin of a patient, if the device or probe is too warm, the probe may be uncomfortable against the skin of the patient. Therefore, the shut-off temperature may be a temperature that has been calculated and pre-programmed to be the maximum temperature at which the probe can be used on the skin of a patient. Once the probe reaches this temperature it may automatically shut-off because use of the probe at a warmer temperature may cause discomfort to the patient.

As shown in FIG. 8, while the probe is at rest after being used, the probe may cool down. When the probe is not in use and is not actively charging, the probe will not generate heat, and so the probe should cool towards ambient temperature. While the probe is at rest and has been resting long enough to reach ambient temperature, the probe will stay at ambient temperature until it is used or charged.

Charging the device may cause the probe to generate heat. If the probe is at ambient temperature and then is charged, the probe will reach a temperature higher than ambient while charging. The charging temperature may be below the shut-off temperature, but above ambient temperature. If the probe has been used recently, such that it has generated heat and then is charged, the probe may cool down from being at rest, but also generate heat from being charged, such that the probe may cool down while charging, but at a slower rate than the probe would cool down at rest.

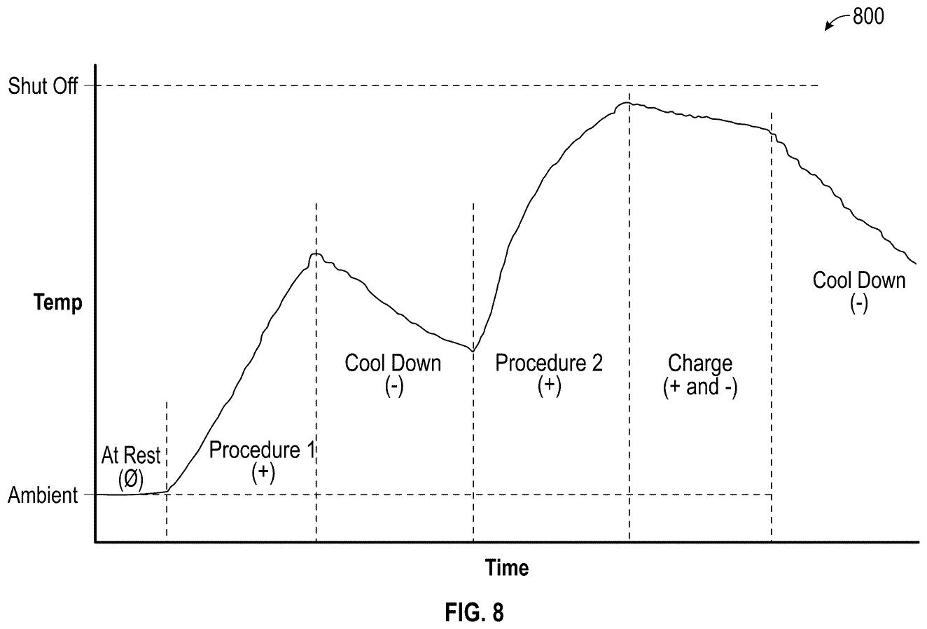

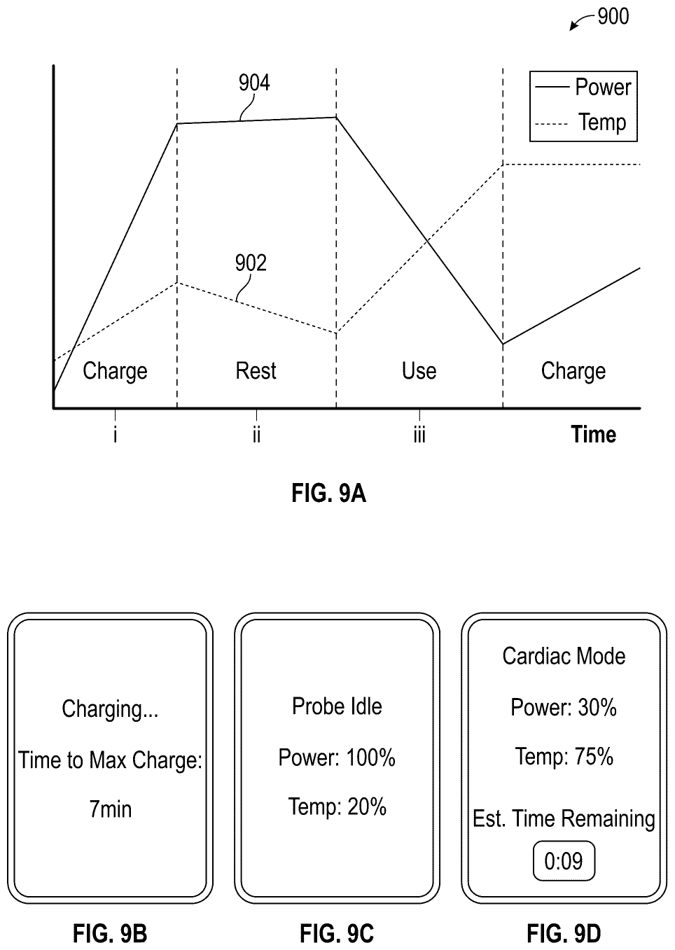

FIG. 9A shows an example plot 900 of temperature-vs-time 902 and power-vs-time 904 for a probe while charging, while at rest, and while in use. This plot 900 illustrates how charging the probe increases the temperature and the power, how keeping the probe at rest may decrease the probe's temperature while retaining the same power level, how using the probe may cause an increase in the probe's temperature and a decrease in the probe's power over time, and how charging the probe may increase the probe's power and cause the probe to retain heat.

FIGS. 9B-9D show various user interfaces, corresponding to different points in time on the plot 900. The user interface may be able to display to the user what state the probe is currently in and the probe's current power levels and temperature. Power levels, for example, may be displayed in terms of percentages, such as full power being 100% and out of power having completely drained the battery being 0%. The temperature may also be displayed in percentages, such as a percentage of the maximum temperature the probe has been programmed to reach.

For example, the range of temperature may start at 0% at ambient temperature and go up to 100% at the maximum/shut off temperature. The UI may also show other calculated information, such as how long it should take the probe to finish charging its battery, or how long the probe should be able to operate at its current settings before overheating or running out of power.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A method for estimating operational capabilities of an ultrasound imaging device, the method comprising: at a computing device that includes one or more processors and memory associated with the ultrasound imaging device: receiving a selection of a medical procedure; determining an operating parameter of the imaging device based on the selected medical procedure; receiving operational data from a sensor of the imaging device; calculating a capability estimation, related to the selected medical procedure to be performed using the imaging device, that is based on the operating parameter and the operational data; and outputting the capability estimation to a user.

Clause 2. The method of Clause 1, wherein the sensor comprises an internal temperature sensor.

Clause 3. The method of any of the preceding Clauses, wherein the sensor comprises an ambient temperature sensor, and the operational data is based on an ambient temperature reading from the ambient temperature sensor.

Clause 4. The method of any of the preceding Clauses, further comprising receiving a mode input based on a desired operating mode of the imaging device.

Clause 5. The method of any of the preceding Clauses, wherein the outputting the capability estimation is performed before the imaging device is used to perform the medical procedure.

Clause 6. The method of any of the preceding Clauses, wherein the outputting the capability estimation updates while the imaging device is used to perform the medical procedure.

Clause 7. The method of any of the preceding Clauses, wherein the receiving a selection comprises presenting a plurality of medical procedures from which the medical procedure is selected.

Clause 8. The method of any of the preceding Clauses, wherein the determining the operating parameter comprises determining at least one of a signal-to-noise ratio (SNR) of the imaging device, a temperature profile of the imaging device, a scan run time, a maximum operating temperature of the imaging device, a cool-down time, a battery power consumption rate of the imaging device, a battery power level of the imaging device, a battery power level of the electronic device, a battery power consumption rate of an electronic device used in conjunction with the imaging device, a minimum power level of the imaging device, a minimum power level of the electronic device, a battery recharge rate of the imaging device, or a battery recharge rate of the electronic device.

Clause 9. The method of any of the preceding Clauses, wherein the receiving operational data comprises receiving a temperature reading of the imaging device.

Clause 10. The method of any of the preceding Clauses, wherein the calculating comprises inputting the operational capability and the selected medical procedure into an algorithm.

Clause 11. The method of any of the preceding Clauses, wherein the outputting comprises providing an indication of whether the imaging device can complete the selected medical procedure.

Clause 12. The method of any of the preceding Clauses, further comprising receiving a mode input based on the selected medical procedure.

Clause 13. The method of any of Clauses 1-11, further comprising receiving a mode input selected from a plurality of available excitation modes in which the imaging device can operate.

Clause 14. The method of Clause 13, wherein the excitation modes correspond to different imaging capabilities of the imaging device.

Clause 15. The method of Clause 13 or 14, wherein the excitation modes use different amounts of available power of the imaging device.

Clause 16. The method of any one of Clauses 13-15, wherein the excitation modes generate different amounts of heat when operated in.

Clause 17. The method of any of the preceding Clauses, wherein the imaging device comprises a battery.

Clause 18. The method of Clause 17, wherein the battery is rechargeable.

Clause 19. The method of Clause 17 or 18, wherein the sensor comprises a temperature sensor for the battery.

Clause 20. The method of Clause 19, wherein the operational data is based on an internal temperature reading from the imaging device.

Clause 21. The method of Clause 19 or 20, wherein the operational data is based on a component temperature reading from an electrical component of the imaging device.

Clause 22. The method of any one of Clauses 19-21, wherein the operational data is based on an internal temperature reading from a transducer array of the imaging device.

Clause 23. The method of any of the preceding Clauses, further comprising receiving an estimate of how long the imaging device needs to be in a rest state before the imaging device can be used to complete the medical procedure.

Clause 24. The method of Clause 23, wherein the rest state comprises a state in which the imaging device is cooling and is not in use

Clause 25. The method of Clause 23 or 24, wherein the rest state comprises a state in which the imaging device is charging.

Clause 26. The method of any of the preceding Clauses, wherein the method further comprises: determining a second operating parameter of a second imaging device based on the selected medical procedure; receiving second operational data from a sensor of the second imaging device; calculating a second capability estimation, related to the selected medical procedure to be performed using the second imaging device, that is based on the second operating parameter and the second operational data; and outputting the second capability estimation to the user.

Clause 27. The method of Clause 26, further comprising receiving an estimate of how long the second imaging device needs to be in a rest state before the second imaging device can be used to complete the medical procedure.

Clause 28. The method of any of the preceding Clauses, wherein the outputting the capability estimation comprises displaying the capability estimation on a display.

Clause 29. The method of Clause 28, wherein the display comprises an estimated time of use remaining for the imaging device.

Clause 30. The method of Clause 28 or 29, wherein the display comprises an estimated battery power remaining for the imaging device.

Clause 31. The method of any one of Clauses 28-30, wherein the display comprises an estimated time remaining before repair of the imaging device is needed.

Clause 32. The method of any of the preceding Clauses, wherein the receiving the selection comprises receiving a selected series of medical procedures.

Clause 33. The method of Clause 32, wherein the calculating a capability estimation includes a consideration of each medical procedure in the series.

Clause 34. The method of Clause 33, wherein the calculating a capability estimation includes determining a prescribed order of each medical procedure in the series.

Clause 35. The method of any one of Clauses 32-34, wherein the outputting the capability estimation comprises displaying the capability estimation on a display.

Clause 36. The method of Clause 35, wherein the outputting the capability estimation includes a forecast of which of the series of medical procedures the imaging device will be able to complete.

Clause 37. The method of Clause 36, wherein the display comprises the forecast of which of the series of medical procedures the imaging device will be able to complete.

Clause 38. A method for estimating replacement time for a component of an ultrasound imaging device, the method comprising: at a computing device that includes one or more processors and memory associated with the ultrasound imaging device: determining an operating parameter of the component; receiving operational data from a sensor of the imaging device, the operational data being representative of the operating parameter of the component; calculating a service estimation based on the operating parameter and the operational data; and outputting the service estimation to a user for assisting in replacement or repair of the component of the imaging device.

Clause 39. The method of Clause 38, wherein the determining the operating parameter comprises determining at least one of a signal-to-noise ratio (SNR) of the imaging device, a temperature profile of the imaging device, a scan run time, a maximum operating temperature of the imaging device, a cool-down time, a battery power consumption rate of the imaging device, a battery power level of the imaging device, a battery power level of the electronic device, a battery power consumption rate of an electronic device used in conjunction with the imaging device, a minimum power level of the imaging device, a minimum power level of the electronic device, a battery recharge rate of the imaging device, or a battery recharge rate of the electronic device.

Clause 40. The method of Clause 38 or 39, wherein the operational data is indicative of at least one aspect of a current performance of the imaging device.

Clause 41. The method of any one of Clauses 38-40, wherein the determining the operating parameter comprises receiving a selection of a medical procedure and basing the operating parameter on the selected medical procedure.

Clause 42. The method of any one of Clauses 38-41, wherein the service estimation comprises an estimated time remaining for use of the component and/or an indication of whether to replace the component.

Clause 43. The method of any one of Clauses 38-42, further comprising shutting down an operation of the imaging device based on the service estimation.

Clause 44. A system for predicting whether an electronic medical imaging device will be able to complete a certain task, the system comprising: a medical imaging device comprising a housing configured to carry internal electrical components, a plurality of temperature sensors, and a circuitry configured to permit the imaging device to operate in a plurality of operational modes; an algorithm capable of receiving input from the imaging device; a processor and memory for performing the algorithm based on data from the imaging device and a given operational mode; and a display capable of showing a capability estimation based on the algorithm.

Clause 45. The system of Clause 44, wherein the plurality of operational modes corresponds to different medical procedures that the imaging device can perform.

Clause 46. The system of Clause 44 or 45, wherein the capability estimation comprises a prediction of whether the imaging device will be able to complete a medical procedure.

Clause 47. The system of any one of Clauses 44-46, wherein the capability estimation comprises a prediction of whether the imaging device will be able to complete a series of medical procedures.

Clause 48. The system of any one of Clauses 44-47, wherein the capability estimation comprises a prediction of whether the imaging device will be able to complete a test, diagnostic procedure, or exploratory scan.

Clause 49. The system of any one of Clauses 44-48, wherein the display allows a user of the imaging device to select one of the plurality of operational modes.

Clause 50. The system of any one of Clauses 44-49, wherein the data from the imaging device on which the algorithm bases the capability estimation comprises (i) one or more temperature readings from the plurality of temperature sensors and (i) the operational mode of the medical device.

Clause 51. The system of any one of Clauses 44-50, wherein one of the plurality of temperature sensors measures an internal temperature of the imaging device.

Clause 52. The system of any one of Clauses 44-51, wherein one of the plurality of temperature sensors measures an ambient temperature.

Clause 53. The system of any one of Clauses 44-52, wherein the internal electrical components of the medical device comprise a transducer array.

Clause 54. The system of any one of Clauses 44-53, wherein the internal electrical components of the medical device comprise a battery.

Clause 55. The system of Clause 54, wherein the battery can be wirelessly charged.

Clause 56. The system of Clause 54 or 55, wherein one of the plurality of temperature sensors measures a temperature of the battery.

Clause 57. The system of any one of Clauses 44-56, wherein the processor is configured to calculate a heating curve and a cooling curve to predict whether the imaging device will be able to complete the task.

Clause 58. An ultrasound imaging system comprising: one or more medical imaging devices each having a processor and a sensor; and a controller device having a program stored in memory, the program comprising instructions for: receiving a selection of one or more medical procedures; determining an operating parameter of each of the one or more medical devices based on the selection of one or more medical procedures; receiving operational data from the sensor of each of the one or more medical devices; calculating a capability estimation, related to the selection of one or more medical procedures to be performed using the one or more medical devices, that is based on the operating parameter and the operational data of each of the one or more medical devices; and outputting the capability estimation to a user.

Clause 59. The system of Clause 58, wherein the determining the operating parameter comprises determining at least one of a signal-to-noise ratio (SNR) of the imaging device, a temperature profile of the imaging device, a scan run time, a maximum operating temperature of the imaging device, a cool-down time, a battery power consumption rate of the imaging device, a battery power level of the imaging device, a battery power level of the electronic device, a battery power consumption rate of an electronic device used in conjunction with the imaging device, a minimum power level of the imaging device, a minimum power level of the electronic device, a battery recharge rate of the imaging device, or a battery recharge rate of the electronic device.

Clause 60. The system of Clause 58 or 59, wherein the controller is configured to coordinate a treatment protocol for a single patient or for multiple patients in which the one or more medical procedures is performed using a plurality of medical devices.

Clause 61. The system of any of Clauses 58-60, wherein the imaging device comprises a plurality of operational modes, and the calculating the capability estimation is also based on an operational mode of each of the one or more imaging devices.

Clause 62. The system of any one of Clause 58-61, wherein the one or more medical devices comprises one or more ultrasound imaging probes.

Clause 63. The system of any one of Clauses 58-62, wherein the one or more medical procedures comprises a plurality of imaging scans.

Clause 64. A system for avoiding downtime during a medical imaging scan, the system comprising one or more medical imaging devices comprising a sensor, a plurality of operational modes, and an operational shut-off limit causing the one or more medical imaging devices to cease operation in response to the sensor, wherein a system processor and a memory are configured to receive a reading from the sensor and perform an algorithm to provide a real-time operational capability prediction of the one or more medical imaging devices based on the reading, a selected operational mode, and a selected medical procedure.

Clause 65. The system of Clause 64, further comprising a means for displaying the real-time operational capability prediction of the one or more medical imaging devices.

Clause 66. The system of Clause 64 or 65, wherein the operational capability prediction comprises a calculated answer as to whether the medical imaging device can complete the medical imaging scan.

Clause 67. The system of any one of Clauses 64-66, wherein the shut-off limit comprises a maximum allowable temperature of the one or more medical imaging devices.

Clause 68. The system of any one of Clauses 64-67, wherein the shut-off limit comprises a battery power level of the one or more medical imaging devices.

Clause 69. The system of any one of Clauses 64-68, wherein the sensor comprises a temperature sensor.

Clause 70. The system of any one of Clauses 64-69, wherein the sensor comprises a battery power sensor.

Clause 71. The system of any one of Clauses 64-70, wherein the system processor and memory are further configured to perform the algorithm based on at least one of a signal-to-noise ratio (SNR) of the imaging device, a temperature profile of the imaging device, a scan run time, a maximum operating temperature of the imaging device, a cool-down time, a battery power consumption rate of the imaging device, a battery power level of the imaging device, a battery power level of the electronic device, a battery power consumption rate of an electronic device used in conjunction with the imaging device, a minimum power level of the imaging device, a minimum power level of the electronic device, a battery recharge rate of the imaging device, or a battery recharge rate of the electronic device.

Clause 72. The system of any one of Clauses 64-71, wherein one of the plurality of operational modes of the one or more medical imaging devices comprises a battery-saving mode in which a frame rate is lower than an operational mode that is not a battery-saving mode.

Clause 73. The system of any one of Clauses 70-72, wherein the one or more medical imaging devices will automatically switch into a battery-saving mode once the one or more medical imaging devices reaches a pre-determined temperature.

Clause 74. The system of any one of Clauses 64-73, wherein the processor computes a diagnostic prediction of how the one or more medical imaging devices should run and compares this computation to the real-time predictions of the medical imaging device's operational capabilities.

Clause 75. The system of Clause 74, wherein the diagnostic prediction and the real-time prediction are displayed to a user of the medical imaging device on the display.

Clause 76. The system of any one of Clauses 64-75, wherein the one or more medical imaging devices comprises a plurality of medical imaging devices.

Clause 77. The system of Clause 76, wherein a single medical procedure may be started using a first medical imaging device and completed using a second medical imaging device.

Clause 78. The system of Clause 76 or 77, wherein when the operational capability prediction estimates whether a first of the plurality of medical imaging devices will be able to complete the medical procedure before reaching the shut-off limit.

Clause 79. The system of Clause 78, wherein the operational capability prediction further estimates whether a second of the plurality of medical imaging devices could complete the medical procedure.

Clause 80. A non-transitory computer readable storage medium storing one or more programs that, when executed by a computing device having one or more processors and memory, cause the computing device to perform operations comprising: receiving a selection of a medical procedure; determining an operating parameter of the imaging device based on the selected medical procedure; receiving operational data from a sensor of the imaging device; calculating a capability estimation, related to the selected medical procedure to be performed using the imaging device, that is based on the operating parameter and the operational data; and outputting the capability estimation to a user.

Clause 81. The computer readable storage medium further comprising any of the steps, features, or operations of the preceding Clauses.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

As used herein, the term “about” is relative to the actual value stated, as will be appreciated by those of skill in the art, and allows for approximations, inaccuracies and limits of measurement under the relevant circumstances. In one or more aspects, the terms “about,” “substantially,” and “approximately” may provide an industry-accepted tolerance for their corresponding terms and/or relativity between items, such as a tolerance of from less than one percent to ten percent of the actual value stated, and other suitable tolerances.

As used herein, the term “comprising” indicates the presence of the specified integer(s), but allows for the possibility of other integers, unspecified. This term does not imply any particular proportion of the specified integers. Variations of the word “comprising,” such as “comprise” and “comprises,” have correspondingly similar meanings.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

What is claimed is:

1. A method for estimating operational capabilities of an ultrasound imaging device, the method comprising:
at a computing device that includes one or more processors and memory associated with the ultrasound imaging device:
receiving a selection of a medical procedure;
determining an operating parameter of the imaging device based on the selected medical procedure;
receiving operational data from a sensor of the imaging device;
calculating a capability estimation, related to the selected medical procedure to be performed using the imaging device, that is based on the operating parameter and the operational data; and
outputting the capability estimation to a user.

2. The method of claim 1, further comprising receiving a mode input based on a desired operating mode of the imaging device.

3. The method of claim 1, wherein the outputting the capability estimation updates while the imaging device is used to perform the medical procedure.

4. The method of claim 1, wherein the receiving operational data comprises receiving a temperature reading of the imaging device.

5. The method of claim 1, wherein the calculating comprises inputting the operational capability and the selected medical procedure into an algorithm.

6. The method of claim 1, wherein the outputting comprises providing an indication of whether the imaging device can complete the selected medical procedure.

7. The method of claim 1, further comprising receiving an estimate of how long the ultrasound imaging device needs to be in a rest state before the imaging device can be used to complete the medical procedure.

8. The method of claim 1, wherein the method further comprises:
determining a second operating parameter of a second imaging device based on the selected medical procedure;
receiving second operational data from a sensor of the second imaging device;
calculating a second capability estimation, related to the selected medical procedure to be performed using the second imaging device, that is based on the second operating parameter and the second operational data; and
outputting the second capability estimation to the user.

9. The method of claim 8, further comprising receiving an estimate of how long the second imaging device needs to be in a rest state before the second imaging device can be used to complete the medical procedure.

10. The method of claim 1, wherein the outputting the capability estimation comprises displaying the capability estimation on a display.

11. The method of claim 1, wherein the receiving the selection comprises receiving a selected series of medical procedures.

12. The method of claim 11, wherein the calculating a capability estimation includes a consideration of each medical procedure in the series.

13. A method for estimating replacement time for a component of an ultrasound imaging device, the method comprising:
at a computing device that includes one or more processors and memory associated with the ultrasound imaging device:
determining an operating parameter of the component;
receiving operational data from a sensor of the imaging device, the operational data being representative of the operating parameter of the component;
calculating a service estimation based on the operating parameter and the operational data; and
outputting the service estimation to a user for assisting in replacement or repair of the component of the imaging device.

14. The method of claim 13, wherein the determining the operating parameter comprises determining at least one of a signal-to-noise ratio (SNR) of the imaging device, a temperature profile of the imaging device, a scan run time, a maximum operating temperature of the imaging device, a cool-down time, a battery power consumption rate of the imaging device, a battery power level of the imaging device, a battery power level of the ultrasound imaging device, a battery power consumption rate of an electronic device used in conjunction with the imaging device, a minimum power level of the imaging device, a minimum power level of the electronic device, a battery recharge rate of the imaging device, or a battery recharge rate of the electronic device.

15. The method of claim 13, wherein the determining the operating parameter comprises receiving a selection of a medical procedure and basing the operating parameter on the selected medical procedure.

16. The method of claim 13, wherein the service estimation comprises an estimated time remaining for use of the component and/or an indication of whether to replace the component.

17. An ultrasound imaging system comprising:
one or more medical imaging devices each having a processor and a sensor; and
a controller device having a program stored in memory, the program comprising instructions for:
receiving a selection of one or more medical procedures;
determining an operating parameter of each of the one or more medical devices based on the selection of one or more medical procedures;
receiving operational data from the sensor of each of the one or more medical devices;
calculating a capability estimation, related to the selection of one or more medical procedures to be performed using the one or more medical devices, that is based on the operating parameter and the operational data of each of the one or more medical devices; and
outputting the capability estimation to a user.

18. The system of claim 17, wherein the determining the operating parameter comprises determining at least one of a signal-to-noise ratio (SNR) of each of the one or more imaging devices, a temperature profile of each of the one or more imaging devices, a scan run time, a maximum operating temperature of each of the one or more imaging devices, a cool-down time for each of the one or more imaging devices, a battery power consumption rate of each of the one or more imaging devices, a battery power level of the imaging device, a battery power level of the ultrasound imaging device, a battery power consumption rate of an electronic device used in conjunction with one or more of the one or more imaging devices, a minimum power level of each of the one or more imaging devices, a minimum power of the electronic device, a battery recharge rate of each of the one or more imaging devices, or a battery recharge rate of the electronic device.

19. The system of claim 17, wherein the controller is configured to coordinate a treatment protocol for a single patient or for multiple patients in which the one or more medical procedures is performed using a plurality of medical devices.

20. The system of claim 17, wherein the imaging device comprises a plurality of operational modes, and the calculating the capability estimation is based on an operational mode of each of the one or more imaging devices.
